(19) United States
(12) Patent Application Publication
(10) Pub. No.: US 2023/0307116 A1
(43) Pub. Date: Sep. 28, 2023

(54) METHOD AND SYSTEM FOR REPRESENTATION AND ASSESSMENT OF VISUAL PERCEPTUAL, VISUAL MOTOR, AND NEUROPSYCHOLOGICAL FUNCTION

(71) Applicant: Tejaswini Mahulikar, Tampa, FL (US)

(72) Inventor: Tejaswini Mahulikar, Tampa, FL (US)

(21) Appl. No.: 18/170,558

(22) Filed: Feb. 17, 2023

Related U.S. Application Data
(60) Provisional application No. 63/311,124, filed on Feb. 17, 2022.

(51) Int. Cl. G16H 20/70; G16H 50/20

(52) U.S. Cl. CPC G16H 20/70; G16H 50/20

(57) ABSTRACT

The present invention concerns a new method and system for representation, measurement, and analysis of visual-cognitive function, more specifically, visual processing, motor, and neuropsychological integration; as well as non-verbal and non-auditory intelligence in humans. This invention seeks to provide a comprehensive and cost-effective process to screen users for the said functions and offer valuable insight into their thought process to identify and design remedies for visual-cognitive and neurodevelopmental deficiencies, especially in children. This new method offers a technologically advanced alternative to commonly used VMI assessments by enabling the collection of a new, more comprehensive set of data points using a digital medium. The invention also lays out a scalable computational system for analysis, inference, and prediction of visual-cognitive function using modern machine learning techniques that can identify salient features in the data and define new classifications, offering a powerful tool for researchers in the field of psychology.

### CROSS-REFERENCE TO RELATED APPLICATIONS

This application is a continuation of U.S. Provisional Patent Application No. 63/311,124, filed on Feb. 17, 2022, the entire disclosure of which is incorporated herein by reference.

### STATEMENT REGARDING FEDERALLY SPONSORED RESEARCH OR DEVELOPMENT (IF APPLICABLE)

“Not Applicable”

### FIELD OF THE INVENTION

The present invention relates generally to the field of software-based systems for analyzing psychometric data to assess visual processing and psychomotor function in humans.

### BACKGROUND OF THE INVENTION

Visual processing is the ability to perceive, analyze, synthesize and include the ability to store and recall visual representations. Perception, internalization, and reproduction of visual stimuli contribute to the learning and educational process, and is especially important in early education. In older adults, recalling visual representations, forming visuospatial correlations and constructional praxis contributes to the quality of life. Visual cognitive abilities vary across individuals (Ackerman, 1988; Ackerman, 1989; Cavanagh, 2011; Lu et al. 2011; Fougnie et al. 2012; Critten et al. 2018), as they understand and interpret differently based on their respective abilities to perceive and internalize visual stimuli.

Several neurodevelopmental deficiencies known today are related to visual perceptual, motor, and neuropsychological integration, and problem-solving functions. Neuropsychologists ascribe these functions to different parts of the brain (neuropsychological localization) (Tonkongy and Puente, 2009). For example, the right hemisphere and the motor cortex opposite the dominant hand control visual and motor functions, and the brainstem is associated with visual-motor integration. A lack of development, injury or deterioration in these areas may affect visual perceptual, motor, and neuropsychological functions and/or their integration in an individual.

These deficits may cause learning disabilities in children, which if left unchecked, can lead to failure at school as characterized by challenges in one's ability to read and comprehend written content and/or solve problems that require visual-spatial interpretation. Visual perceptual impairment may also present itself as a result of Dyslexia, brain injury (congenital or acquired), post-operative recovery after neuroskeletal, spinal, or brain surgery, DCD (developmental coordination disorder), Cerebral Palsy, Vision impairment, and Autism Spectrum Disorder (visual-motor aspect). Timely diagnosis of these disorders is critical to provide early and effective intervention. A common example of such a disorder is Dyslexia, which is estimated to affect about 510% of the population. The National Center for Education Statistics reports that 13-17% of the students enrolled in public schools in the U.S. have learning disabilities of various kinds and 19% of adults aged 16-65 scored below level 1 literacy.

According to WHO, around 50 million people worldwide have dementia, and there are nearly 10 million new cases every year. Alzheimer's disease is the most common form of dementia and may contribute to 60-70% of cases. Dementia is one of the major causes of disability and dependency among older people worldwide.

According to CDC, the prevalence of subjective cognitive decline (SCD) is 11.1%, or 1 in 9 adults. The prevalence of SCD among adults aged 65 years and older is 11.7% compared to 10.8% among adults 45-64 years of age. The prevalence of SCD is 11.3% among men compared to 10.6% among women.

Approximately one in three veterans referred to outpatient vision rehabilitation has detectable cognitive impairment.

Visual Motor deficits, including unilateral or bilateral weakness, ataxia, spasticity, and loss of complex movement execution due to multiple possible etiologies, can occur during any brain tumor illness, as a postoperative side effect of neurosurgery, or injury.

When diagnosed, a variety of intervention techniques, such as adaptive training, can be used to help individuals with such deficits. Therefore, the availability of reliable, cost effective, and smart systems and processes that can diagnose and report key neuropsychological parameters is essential.

Visual cognitive disabilities are typically identified through paper-based tests. In these paper-based tests (e.g., Beery and Beery, 2013), including the widely used Beery Buktenica developmental test of visual-motor integration (VMI), subjects are given a series of visual forms (patterns) to be replicated on paper using a pencil/pen. These tests are individually administered and analyzed by trained psychologists. In early education settings, this makes these assessments expensive, and so, naturally, they are only made available to students who have been referred due to severe educational concerns. A vast majority of children, in the millions, remain un-evaluated. A cost-effective and easy-to administer assessment tool is, therefore, required. The need is not just for initial assessment, but also for tracking progress in students benefiting from Individualized Education Programs (IEPs).

In paper-based tests conducted by trained practitioners, scoring is based on guidelines derived from anecdotal data (e.g., Beery and Beery, 2013). This requires pattern recognition and matching, a task that can now be readily and rigorously performed by computers. More importantly, these current assessments only provide limited information, as they do not track visual-motor speed, direction and visual-spatial awareness, which have been known to be important to assessment of visual cognitive functions (Ackerman, 1988; Ackerman, 2007).

For example, results from various studies in educational psychology, experimental and applied psychology have found that visual perceptual and perceptual speed is known to influence the significant interactions between perceptual speed and the order of data elements in predicting such areas as vocabulary learning and search performance. Meltzer found significant correlations between perceptual speed and achievement in both reading and arithmetic in 6-8-year-olds (Meltzer, 1982). This cognitive ability was the main predictor for reading comprehension in 7-year-olds. However, her research suggested that there was a stage in the process of learning how to read or do arithmetic in which perceptual speed plays a major role.

This is consistent with the Ackerman model of skill acquisition (Ackerman, 1989). The model divides skill acquisition into three stages. The first is skill acquisition, an understanding of tasks is achieved and general cognitive abilities such as verbal, numerical and figural are most important. In the second stage, performance of the task becomes quicker as learners try out various methods of simplifying or streamlining tasks. This is where the phase perceptual speed has its greatest impact. On the third stage of skill acquisition, performance of tasks becomes automatic, and psychomotor abilities influence performance. Perceptual speed in cognitive ability is defined as “Speed in comparing figures or symbols, scanning to find figures or symbols or carrying out other very simple tasks involving visual perception.” Individuals who score higher on standard tests of perceptual speed perform higher quality searches than those (as measured by standard precision and recall rations) than those who score lower on the tests.

Consequently, for improving existing assessments, there is also a long-felt need for methods that can record and analyze these features. Such methods, however, add further complexity to data analysis thus creating even further hurdles that have not been surmounted with current approaches or technology. What is needed, therefore, is a method and system for representation and assessment of visual perceptual, visual motor and neuropsychological function that resolves or improves upon current methods and systems.

### BRIEF SUMMARY OF THE INVENTION

Learning, particularly in elementary education, and visual cognitive function in general, is largely based on perception and reproduction of visual stimuli, or quite simply, perceiving, internalizing, and copying/imitation of visual information. The fundamental reasoning behind this invention is that an individual's approach and ability to reproduce/copy a given visual form by using their ability to integrate visual and motor functions can be considered as their ability to solve a problem based on visual perceptual information gathered from the visual stimulus by looking at the form. Individuals have varying visual cognitive abilities, and therefore, solve visual problems in unique ways. The system and method in this invention provide an ability to capture, analyze, and report variations and anomalies in these cognitive abilities and functions.

The present invention concerns a new method and system for representation, measurement, and analysis of visual cognitive processes, more specifically, visual perceptual, motor, and neuropsychological integration, as well as problem-solving function, or non-verbal intelligence in humans. It includes a method, as well as a computational system for assessment of current abilities of users, a novel form of measurement and representation of the said functions, and a new reporting mechanism and format. This invention seeks to provide an improved, cost-effective process to screen users for the said visual-cognitive functions to identify and design remedies for cognitive and neurodevelopmental deficiencies associated with said functions.

In one embodiment, the invention relates to a new method of gathering data related to visual cognitive processes, more specifically, visual perceptual, motor, and neuropsychological integration as well as problem-solving ability, or non-verbal and nonauditory intelligence in humans; processing the data using a hybrid computational data processing system that consists of both traditional heuristics-based computation, as well as machine learning techniques, some of which offer the additional advantage of automatic feature discovery or pattern recognition, to analyze the gathered data; and reporting inferences and predictions as results.

### BRIEF DESCRIPTION OF THE DRAWINGS

The present invention may be better understood, and its numerous objects, features, and advantages made apparent to those skilled in the art by referencing the accompanying drawings. The use of the same reference number throughout the several figures designates a like or similar element.

FIG. 1 depicts an example of overall system architecture;

FIG. 2 is a sample graphical user interface rendered by a software application running on a handheld device, henceforth referred to as “client application”, when a subject is presented with a set of visual forms;

FIG. 3a is a sample graphical user interface on the client application when a subject attempts to reproduce a specific visual form. The subject renders a pattern on a touchscreen device using a stylus;

FIG. 3b shows examples of Visual representation of numerical data used for storing drawing speed and touchpoints on a pattern sketched by the subject;

FIG. 3c is an example of numeric data collected internally by the client application on the handheld device as the subject makes the drawing in FIG. 3b;

FIG. 4 shows the variations between subjects in their sketching speeds, direction, touchpoints, which provide critical insights into cognitive function;

FIG. 5 contains an example of a color-coded image generated by the client application;

FIG. 6 illustrates the capabilities and key components of a Remote Data Storage Subsystem;

FIG. 7 illustrates the capabilities of a Remote Data Processing Subsystem showing various analysis approaches, ranging from purely heuristics-based to those based on sophisticated machine learning, as well as mixtures of the two;

FIG. 8 illustrates the basic capabilities of a Remote User Management Subsystem and its interconnectivity with other parts of the system; and

FIG. 9 contains a sample of reported findings for a given task;

### DETAILED DESCRIPTION OF THE INVENTION

A method, a computational system, and computer-usable medium are disclosed for representation, measurement, analysis, and reporting of visual-cognitive function, more specifically, visual, motor, and neuropsychological integration in humans. The computational system also constitutes a computer program product that may include and use computer-readable storage media having computer-readable program instructions thereon for causing a processor to carry out aspects of the present invention.

Any computer-readable storage medium in the system implementing this invention can be a tangible device that can retain and store instructions for use by an instruction execution device. The computer-readable storage medium may be, for example, but not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. Any computer-readable storage medium, as used herein, is not to be construed as being transitory signals per se, such as radio waves or other freely propagating electromagnetic waves, electromagnetic waves propagating through a wave-guide or other transmission media (e.g., light pulses passing through a fiber-optic cable), or electric signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages like JavaScript, Node.JS, and R, also including an object-oriented programming language such as Swift, Python, Smalltalk, C++, Go or the like, and conventional procedural programming languages, such as the “C” programming language or similar programming languages. The computer-readable program instructions may execute entirely on the user's computer, partly on the user's computer, as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or another device to cause a series of operational steps to be performed on the computer, other programmable apparatus, or another device to produce a computer-implemented process, such that the instructions which execute on the computer, other programmable apparatus, or other device implement the functions/acts specified in the flowchart and/or block diagram block or blocks.

The flowchart and block diagrams in the Drawings attachment illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

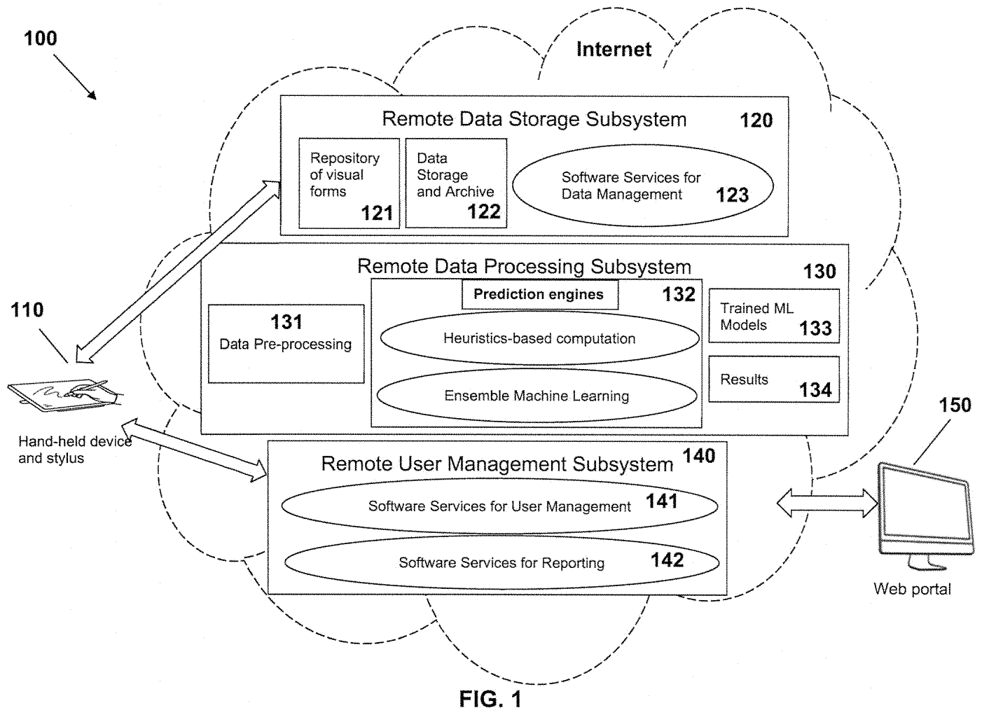

FIG. 1 is an illustration of a generalized computational system that can be used to implement the present invention. The computational system provided in this invention consists of a handheld device 110 that is runs a software client application program that renders a graphical user interface, and measures and collects multi-dimensional data as a subject performs tasks that require visual processing, a data storage and archiving mechanism 120 and a set of computational processors that run software server programs that analyze the captured data and produce inferences or predictions 130. The system also provides a secure mechanism for administrative users to interact with it 140 to perform tasks like registration, the configuration of visual form sets, and retrieval of reports through a Web Portal 150.

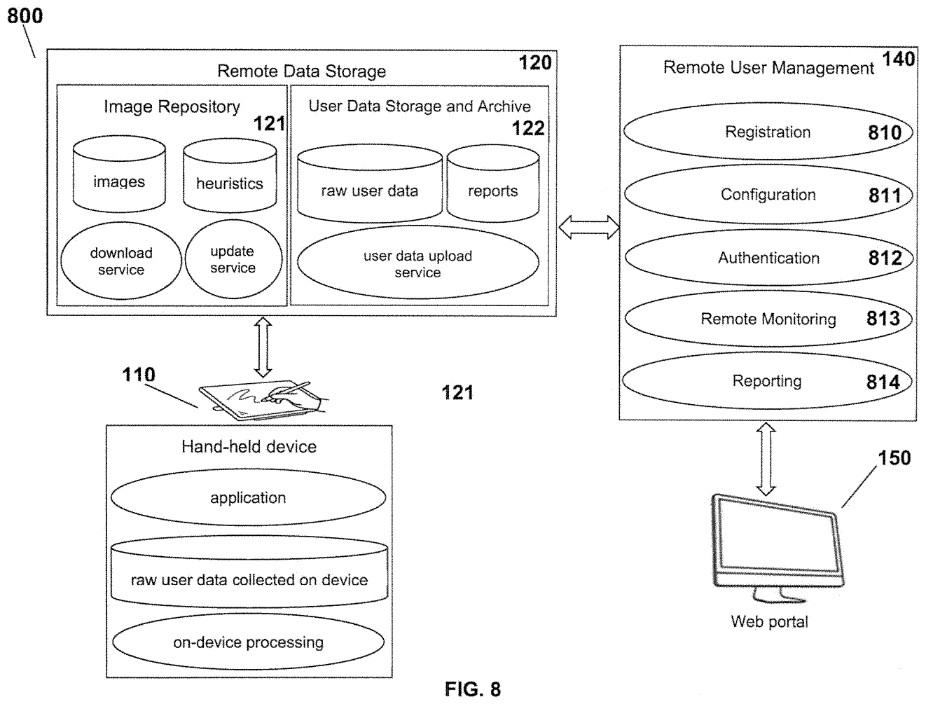

The handheld device 110 in this system can be any digital product with a screen that can run a software client application program that offers an interactive medium in the form of a graphical user interface to draw and can therefore serve as a digital alternative to paper. Examples of such products include but are not limited to digital tablets such as—the iOS based iPad, Android-based Samsung Galaxy Tab, FireOS based Amazon Fire Tablet, Windows-based Surface, or any other tablet or digital interface. A connected digital stylus pen that is compatible with the device can be used, and so can any of the other available soft-tip capacitive stylus products. It provides a means for the subject (user) to view and draw VMI visual patterns. An application running on the device as illustrated in 800 (FIG. 8) renders a graphical user interface that presents the subject with the goal-oriented task of drawing a series of visual forms or patterns.

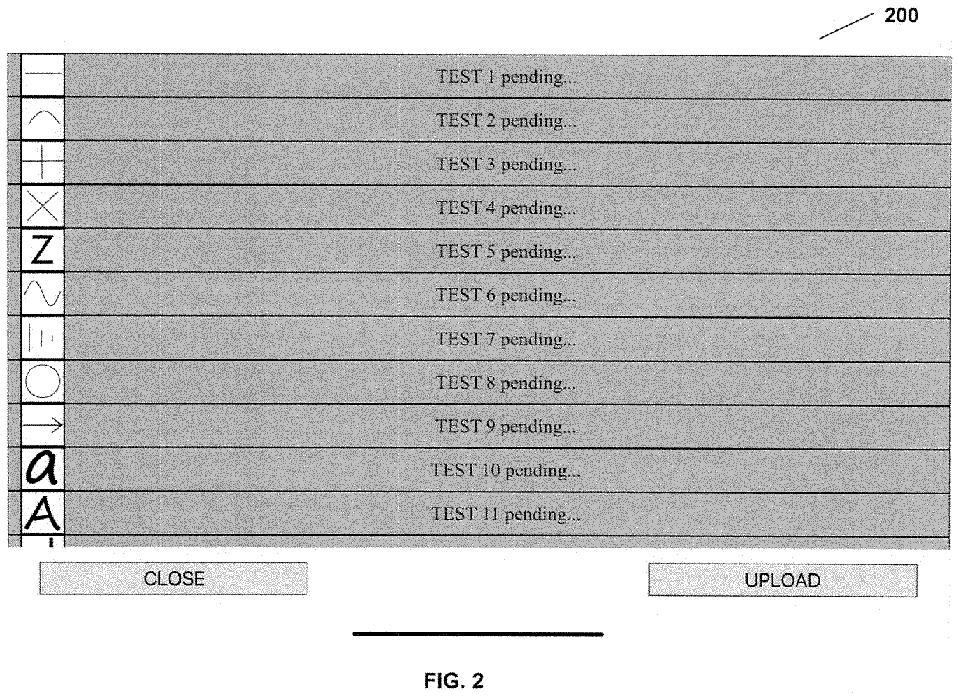

In an embodiment, the forms presented to the subject can be pre-selected by a trained professional, such as on the basis of the subject's age and the purpose of the test. The subject goes through a series of tasks one-by-one and using a stylus draws the image directly on the touch-screen device (FIG. 2). As the subject draws the presented visual form, the application not only keeps track of the final image but also internally tracks other key features missing in paper-based tests, including rendering speed in different parts of the pattern, direction, pressure gradient and touchpoints. These new data features offer valuable insight into the thought process of an individual as they solve a visual problem. The method by which we track theses new features is novel and is a key aspect of this invention.

The multi-dimensional data collected by the client application may include—structured data consisting of numeric measurements, and unstructured data consisting of images and video.

The client application running on the handheld device can be used in online mode when there is internet connectivity, or offline mode when there is no internet connectivity.

In an embodiment, the client application that runs on the handheld device may authenticate an administrative user.
The client application may store the collected raw visual-processing data temporarily on the device, or any other computer-readable storage medium accessible. This would especially be true in the absence of internet connectivity.

120, 130, and 140 are parts of the system that are embodied by a scalable set of remote computer servers that perform different functions utilizing computer-readable storage media and provide services that can be accessed on the network they connect with.

Some of the remote computer servers in 120, 130, and 140 may be collocated on a single hardware computing device or may be distributed across multiple computational devices and storage media connected by a network.

The functionality hosted on these subsystems by computer server programs can be accessed through common software network protocols like FTP, SFTP, SCP, SOAP, and more commonly, HTTP endpoints like REST APIs.

Accessing any of these servers would require a server computer program commonly referred to as “Software Service”. Several software services performing various functions run on the remote server subsystems. For e.g. 132, 141, 142.

The remote computer servers can be implemented using on-premise computation capabilities and infrastructure or can be deployed on any private or public cloud infrastructure using pre-configured or on-demand computational resources.

150 is a web portal through which administrative users can access the system. Any computer connected to the internet or the network in use for the remote subsystems can be used as a web portal. An administrative user can access the system via the web portal to register, retrieve data, and request reports.

FIG. 2 shows an example of the graphical user interface rendered by the client application running on the handheld device where a subject is presented with a pre-configured set of visual forms and asked to reproduce them sequentially using a stylus, thus providing a digital medium that is similar to a paper and pencil.

In an embodiment, the client application uses a set of visual forms installed with the application.

In an embodiment of this invention, the set of visual forms presented to the subject may be configurable through the user management subsystem of servers 140 wherein an administrative user could choose the set of visual forms in advance through a web portal 150.

The client application may download the set of visual forms configured by an administrative user upon authentication from a repository of visual-forms 121 from the remote data storage server subsystem 120.

In another embodiment of the invention, the visual forms can also be presented to the user on paper to accommodate potential physical disabilities in the subject. For each of the visual forms presented, the individual attempts to reproduce the form using a stylus on the device.

As the individual sequentially attempts to draw each visual form presented to him/her, the client application internally records several numeric measurements, image representations, and videos that capture the approach used by the individual.

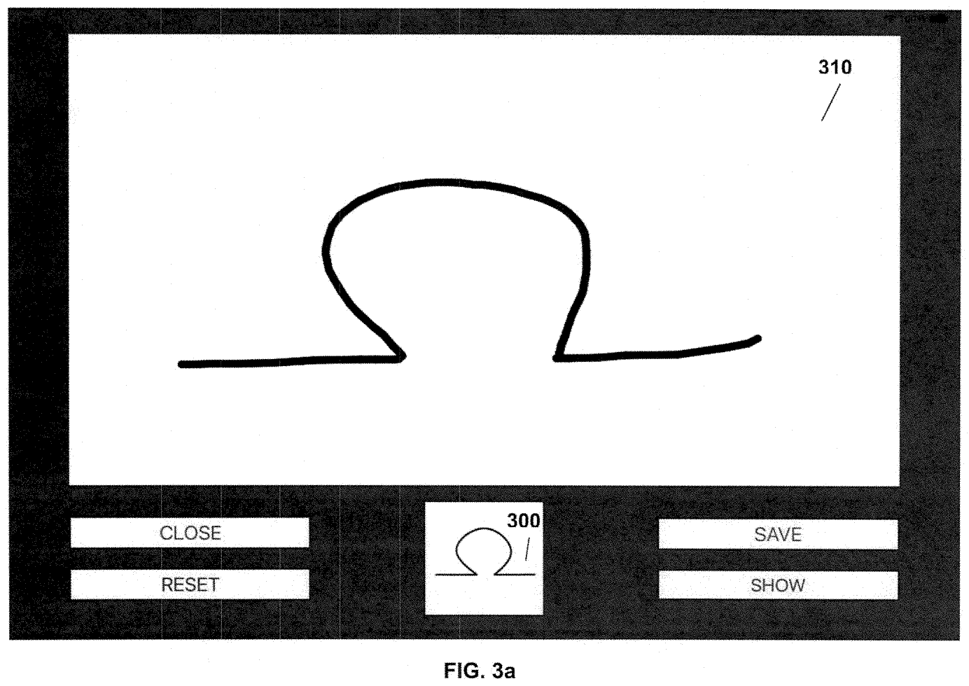

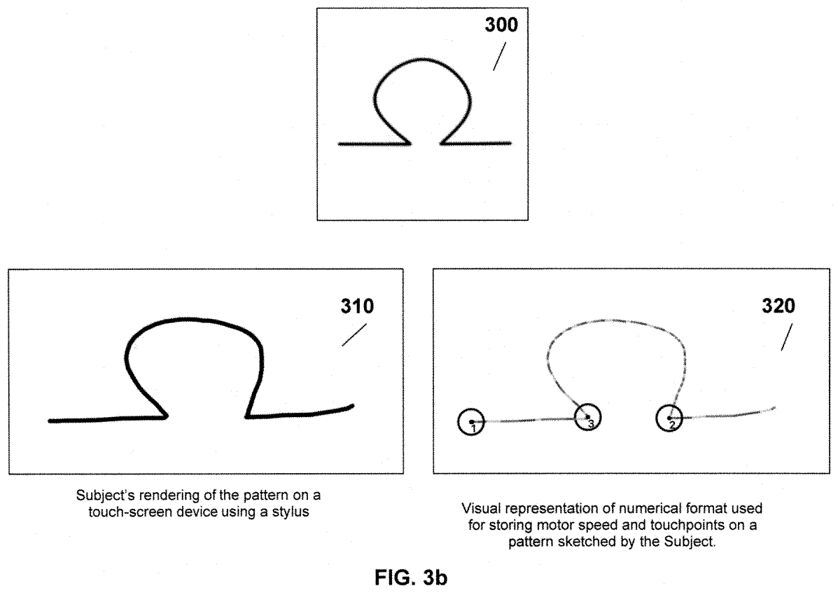

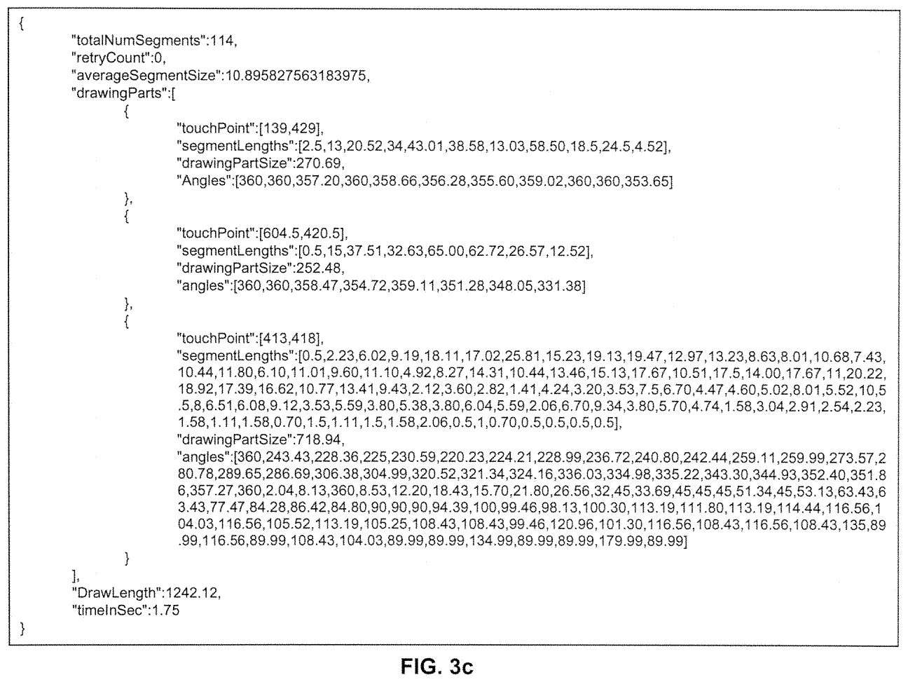

FIG. 3a shows an example of the user interface of the client application running on the handheld device where a subject is presented a graphical user interface with a drawing region 310 to reproduce a specific visual form selected from the set shown in FIG. 2.

The client application internally creates color-coded images that serve as a visual representation of the individual's performance in reproducing the visual form that represents—
relative speed of execution at different parts of the form,
continuity or discontinuity of thought within the form,
containment and enclosure,
Separation of parts,
Spatial awareness (awareness of key spatial relations within the form)
general problem-solving approach

This idea is depicted in FIG. 3b which shows the reproduced visual form drawn by subject 310 and the approach used therein. In 320 the subject used 3 touch points and completed the task using differential motor speeds in different parts of the drawing. The image also indicates the sequence of touch points made by the subject when drawing the form.

The client application records several numeric measurements as the subject attempts to draw the visual form. This psychometric data contains specific numeric measurements such as—time taken to perform the task, number of retries, coordinates of starting points, coordinates of points of discontinuity and resumption, and a representation of the speed of execution between points of discontinuity. This data is used for mathematical analysis to detect anomalies.

In one embodiment, these measurements are saved in a file on the device in any of the known formats. These formats include JSON, XML, CSV, or any other format. FIG. 3c shows the contents of a sample data file containing such measurements in JSON format.

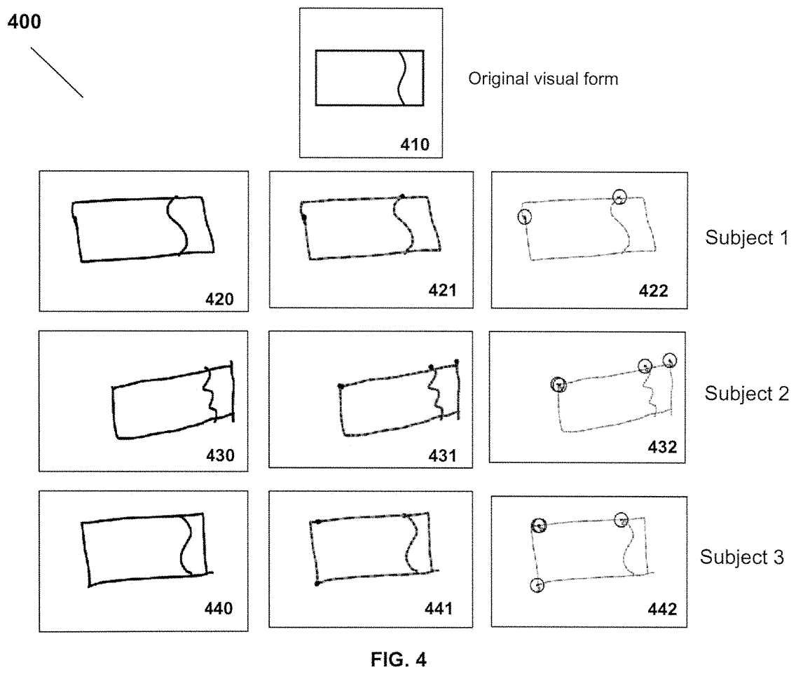

FIG. 4 shows differences in approach and the resulting visual form between 3 individuals as reflected in the color-coded images that show variations in touch points, speed, direction, and the proximity of the reproduced form to the original. Subject 1 used 2 touch points and reversed the orientation of the curved line connecting the two parallel sides of the enclosing rectangle indicating a tendency to laterally invert visual forms. In presence of additional corroborative data in the form of other similar examples, this tendency may indicate dyslexia. Subjects 2 and 3 used 4 touch points each at different parts of the visual form, and only Subject 3 was close to reproducing it accurately.

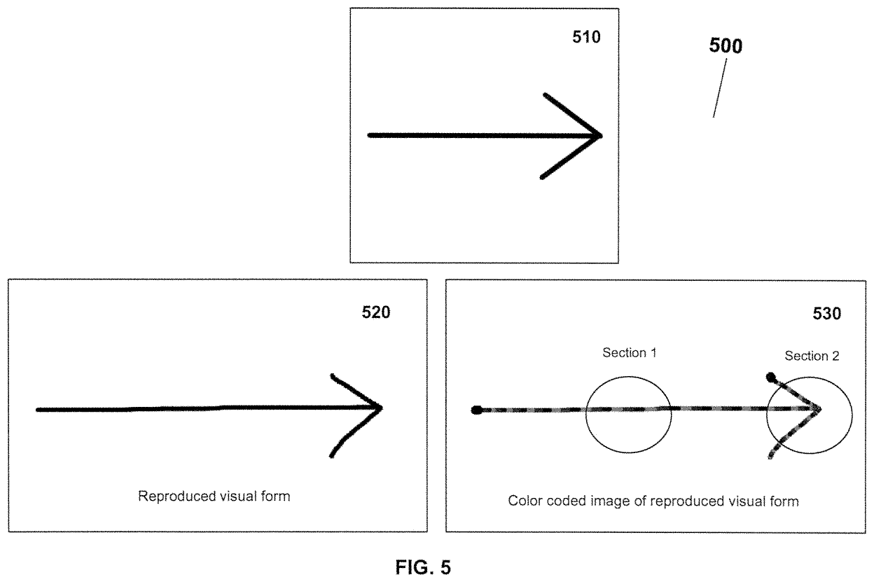

FIG. 5 is another example of a color code representation created by the application. In 530, the speed of execution was greater in section 1 when compared to section 2 as shown by greater color segment lengths in section 1. Longer segment lengths in section 1 when compared to section 2 indicate that the individual drew section 1 faster (greater speed of execution) than section 2. In the example shown, color segments are repeated in the sequence in the order—red, blue, green. Alternate color selections can be made so long as they are consistent with other anecdotal data used for subsequent data-driven analysis.

The client application may generate other encoded images with alternate coding strategies, and perform additional numeric measurements to capture other salient features of the individual's strategy and psychomotor skill, e.g. pressure applied at various points in the drawing can be measured and represented, etc.

The data thus gathered on the device includes a black and white image of the visual form reproduced by the individual, numeric measurements, as well as color-coded images and video.

In an embodiment of this system, a pressure-sensitive stylus pen can be paired with the handheld device to capture pressure gradient measurements as a part of raw data.

In an embodiment of this system, a pressure-sensitive screen on the handheld device may be used to capture pressure gradient measurements as a part of raw data.

This captured data may be stored locally on the device until it is uploaded to the remote data storage subsystem 102 and stored in data storage and archive 122 which essentially constitutes a computer-readable storage medium, and associated software services or computer server programs. The process of data upload from the device can be instantaneous or delayed. In the absence of network connectivity, the data may be stored on the device for a longer duration. The data may also be manually transferred to the remote data storage subsystem using intermediate storage media and computer programs serving the specific purpose.

Since all the raw data collected and processed by the system in this invention is intended to be used in the future to continually optimize and improve the system's inference and prediction capabilities, the data storage server system also can archive data for and retrieve data upon request.

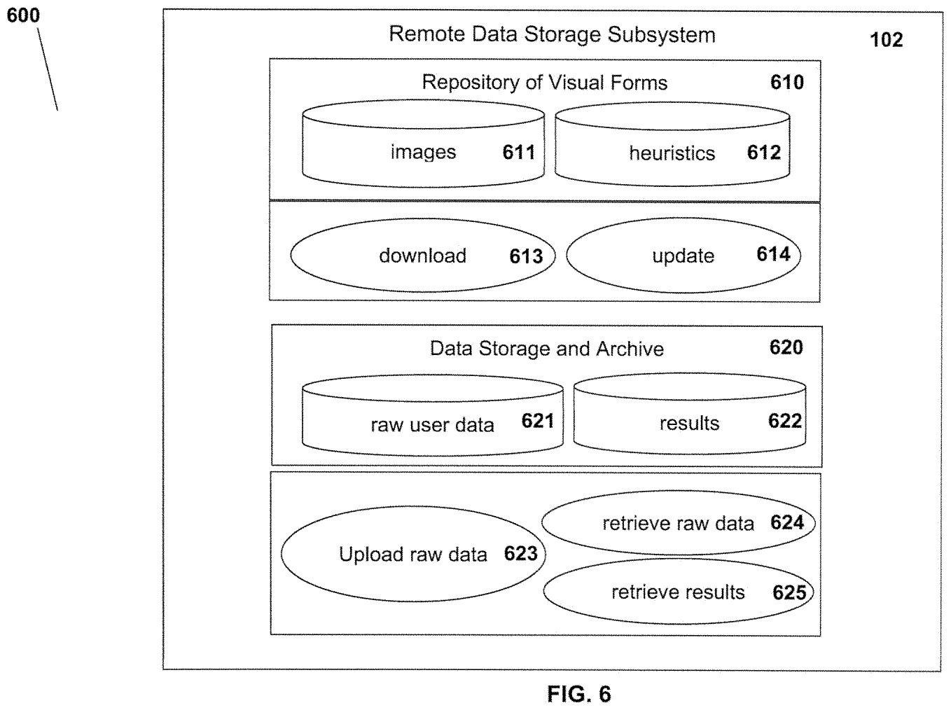

FIG. 6 shows some of the functions supported by the data storage server subsystem. Different functions served by this subsystem may be implemented by a single monolithic computer server program or by multiple scalable distributed server programs. The storage aspect of the functionality may be implemented by any available computer storage medium on-premise or on private or public cloud infrastructure.

The data storage subsystem can store and archive data such as raw visual-cognitive data 621 and post-processing results 622 and retrieve it upon request. New data can be uploaded, and stored data can be retrieved via service endpoints, or computer server programs awaiting such requests 623, 624, 625.

Before processing, the acquired data moves through several data preparation steps like data scaling, normalization, and standardization.

In an embodiment of the present invention, the remote data storage subsystem FIG. 6 maintains a repository of visual forms 610. This repository consists of a collection of images of visual forms that can be used for the assessment of visual-cognitive functions in a subject 611. Each visual form may be associated with a set of heuristics 612, which essentially are anecdotal rules and thresholds created based on historic observations. The performance of a subject in reproducing a given visual form may be computationally evaluated using these heuristics.

In an embodiment of the present invention, an administrative user configures a set of visual forms through the web portal. These visual forms and their associated heuristics may be downloaded by the client application on the handheld device upon authenticating the administrative user. All subjects interacting with the device will then see the configured set of visual forms. In another embodiment, the client application may be able to apply the downloaded heuristics to the gathered data and output a report on the device immediately upon completion of the task.

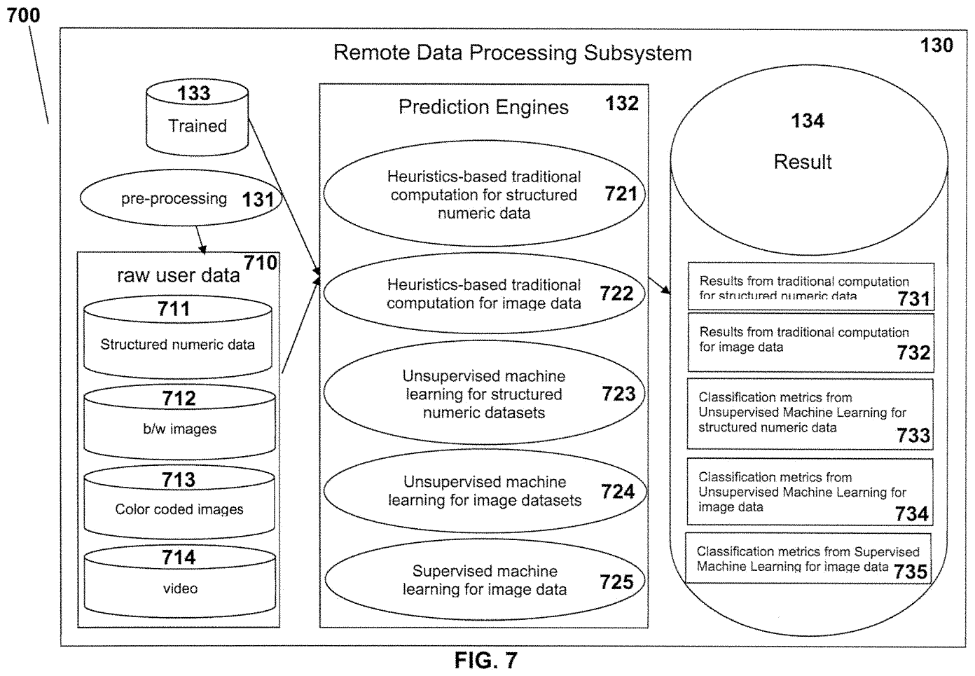

FIG. 7 illustrates a possible implementation of the data processing system 130 in this invention as a set of servers that may run on-premise or on private or public cloud infrastructure. In an embodiment of this invention, new data moves through standard data preparation steps like data scaling, normalization, and standardization before processing 131.

A hybrid computational data processing system that consists of traditional computational programs that are based on heuristics, and more modern machine learning or artificial intelligence-based algorithms may be used for analyzing the data and outputting predictions. These together form the prediction engines 132.

The data collected in this invention is multidimensional and multi-formatted 710. An embodiment of the present invention may implement a hybrid approach for data analysis. It covers multiple concurrent approaches of processing the data using a set of processing engines, each implemented as a separate specialized computer program running on a server, that can either be used in isolation, or in conjunction with other approaches 132.

In an embodiment of this invention, the computational system for data analysis makes inferences based on a traditional computational program that computes prediction output based on structured numeric data measured by the client application 721. Such a program uses anecdotal thresholds to flag data points that do not fall within the threshold values. This embodiment can be used in isolation or in association with other embodiments of this invention that use other data and processing techniques.

In an embodiment, a traditional computational program may be used to compare and evaluate black and white images of drawings made by subjects 722.

In an embodiment of this invention, the system performs predictions using a supervised machine-learning-based service that uses image data captured on the client device 725. This embodiment can be used in isolation or in association with other embodiments of this invention that use other data and processing techniques. Convolutional Neural Networks (CNNs) are frequently used to solve image classification problems. CNN models borrow a great many ideas from historical assessment practices and will continue to do so as the fields of neuroscience and machine learning evolve. However, while analyzing data, and training the CNN model in the present invention, we may continue to find reasons for deviating from the specific guidelines provided by traditional VMI tests.

Semi-supervised learning procedures use the automatic feature discovery capabilities of unsupervised learning systems to improve the quality of predictions in a supervised learning problem. Instead of trying to correlate raw input data with the known outputs, the raw inputs are first interpreted by an unsupervised system. The unsupervised system tries to discover internal patterns within the raw input data, removing some of the noise, and helping to bring forward the most important or indicative features of the data. These distilled versions of the data are then handed over to a supervised learning model, which correlates the distilled inputs with their corresponding outputs to produce a predictive model whose accuracy is generally far greater than that of a purely supervised learning system. This approach can be particularly useful in cases where only a small portion of the available training examples have been associated with known output. Semi-supervised learning allows the system to discover internal patterns within the full set of images and associate these patterns with the descriptive labels that were provided for a limited number of examples. This approach bears some resemblance to our own learning process in the sense that we have many experiences interacting with a particular kind of object, but a much smaller number of experiences in which another person explicitly tells us the name of that object.

In an embodiment, the system performs predictions using an unsupervised machine learning-based service that processes structured numeric data captured by the client application 723. Visual-cognitive factors that do not easily lend themselves to known quantitative parameters can be identified using unsupervised machine learning. This embodiment can be used in isolation or in association with other embodiments of this invention that use other data and processing techniques.

In an embodiment, the system performs predictions using an unsupervised machine learning-based service that processes image data captured by the client application 724.

In another embodiment of this invention, unlabeled structured numeric datasets grouped according to the age of the subject are processed by an Unsupervised Machine Learning algorithm like K-means clustering. In another embodiment, the Unsupervised Machine Learning algorithm is used as a pre-training step. The clusters of datasets (or features discovered) in this step can be used to train a separate processing engine that uses supervised learning algorithms.

In an embodiment of this invention, the system performs predictions using a machine learning-based service that uses video data captured on the client device. This embodiment can be used in isolation or in association with other embodiments of this invention discussed.

In an embodiment, this invention may be dependent on some of the meta-data collected from the subject, like age, gender, and handiness, etc.

In an embodiment of this invention, historic labeled image data may be used to train a supervised machine learning algorithm.

In an embodiment of this system, any archived data, such as images from a completed VMI assessment can be scanned, uploaded to the data storage server, and labeled before processing using the machine learning software and used either in supervised or unsupervised mode to train the models and produce inferences.

In another embodiment of this invention, a computational server running a program that implements algorithms from meta-learning frameworks uses one-shot or few-shot learning where classifiers can be built using very limited amounts of training data.

In an embodiment of this invention uses the “Ensemble” method for processing and analyzing data, which essentially is a machine learning technique that combines different machine-learning models to generate a model with high prediction performance.

Stacking (sometimes called stacked generalization) is a common type of ensemble. It involves training a learning algorithm to combine the predictions of several other learning algorithms. First, all the other algorithms are trained using the available data, then a combiner algorithm is trained to make a final prediction using all the predictions of the other algorithms as additional inputs. Stacking is known to yield better performance than any single one of the trained models. It has been successfully used on both supervised learning tasks and unsupervised learning.

In an embodiment, the results generated from evaluation by each of these processing engines are subsequently aggregated to provide the user with a consolidated report. The reporting service in the present invention consolidates the results generated by the processing engines and runs on a separate set of servers that can be accessed via a web portal.

In an embodiment of this invention, the ensemble machine learning model is deployed as a Web Service (REST API) and can be accessed via the Web Portal.

FIG. 8 illustrates the role of a remote user management server 140 in an embodiment of the present invention where a set of servers that may run on-premise or on private or public cloud infrastructure, allow administrative users to access the system and retrieve data from it. In an embodiment of this invention, an administrative user registers with the system via a web portal.

In an embodiment of this invention, a registered administrative user can configure a set of visual forms that will be downloaded by a client application running on the handheld device in the system upon successful authentication.

In an embodiment of this invention, an administrative user may request reports from the system via the web portal.

In an embodiment of this invention, an administrative user may request any or all of the raw data like video, images, or numeric data captured for a subject. The data can be retrieved from the remote data storage sub-system and sent to the user by various software services in the user management subsystem.

In an embodiment of this invention, an administrative user may request remote monitoring of assessments where the subject's progress can be monitored through a live stream.

In an embodiment of this invention. Inferences and predictions by the different prediction engines can be reported to a user upon request based on the selection of specific processing engine(s) or as a consolidated report containing prediction data from all available engines to allow for comparison.

In an embodiment, captured raw data, specifically images may be included in the report to enable correlation with the predictions and cross-referencing.

In an embodiment of this invention, predictions or characteristics documented in the report include, but are not limited to the following—
Visual Processing and Psychomotor ability
Processing speed and Psychomotor speed
Decision or Reaction Time/Speed
Visual Discrimination
Visual Integrity and efficiency
Problem-solving characteristics
Reversal (lateral and vertical)
Inhibition
Attention
Confidence
General Strategy—directionality
General Strategy—perceptive
Effort
Visual-Spatial Awareness

In an embodiment, a breakdown of the above characteristics may be reported in the form of a set of findings such as, but not limited to—

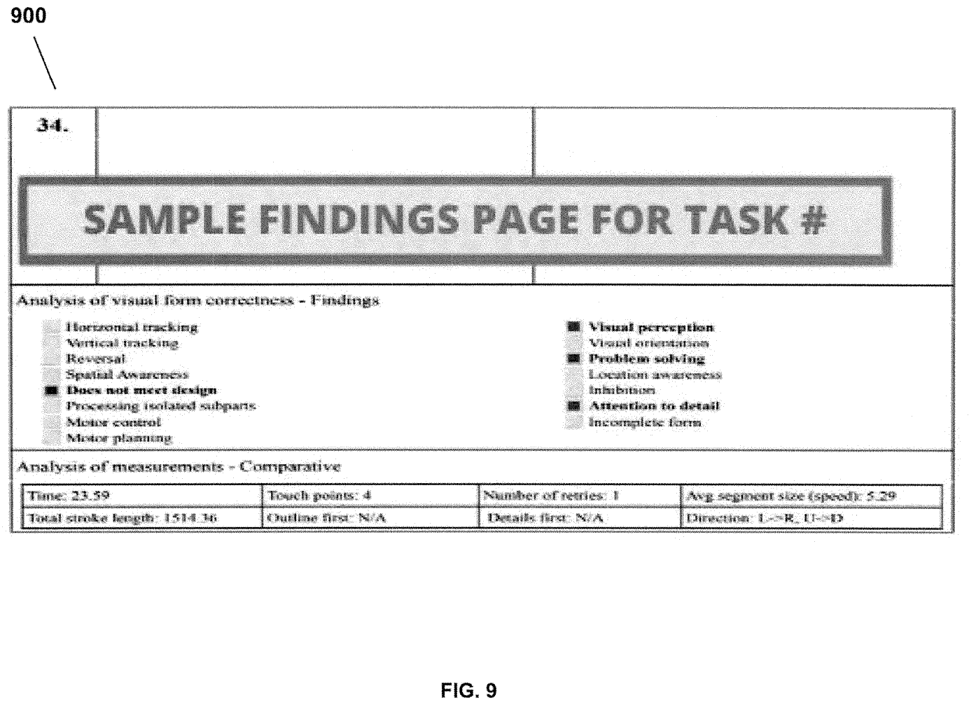

Horizontal tracking, Vertical tracking, Reversal Spatial Awareness, Processing isolated subparts, Motor control, Motor planning, Visual perception, Visual orientation, Problem solving, Visual sequencing, Location awareness, Inhibition, Attention to detail, Incomplete form.
A sample of this embodiment is shown in FIG. 9.

The present invention is unique in that it measures and captures data that has not been measured or considered before—motor speed, direction, visual-spatial awareness, pressure, and general problem-solving approach. It is the first mechanism of its kind that uses powerful computational methods like machine learning that can uncover hidden patterns and improve prediction performance over time.

Over a period of time, as the system processes more data it is expected to undergo continual improvements in the calibration of key measurement factors and their leveling, and therefore, produce more reliable reports. This would enable the present invention to become a standard feature in future psychological assessments.

The data processing capabilities of this invention can also be used to process old archived VMI assessment data recorded in the past. Most of this data is in the form of black and white images that can be processed by using appropriate Machine Learning techniques. This would also result in improvement in the calibration of key measurement factors and their leveling, and therefore, produce more reliable reports over time.

The invention is very relevant in healthcare for seniors for detection of visuospatial impairments as dementia progresses, as well as for assessment and rehabilitation after traumatic brain injury or post neuro-skeletal surgery.

This invention is relevant for detection and subsequent corrective training and therapeutic monitoring for cases of
Dyslexia
brain injury (congenital or acquired)
post-operative recovery after neuro-skeletal, spinal, and/or brain surgery
DCD (developmental coordination disorder)
Cerebral Palsy
Visual impairment
Autism Spectrum Disorder (visual-motor aspect)
Age-related cognitive decline in adults (Dementia, Alzheimer's, other Visioconstructional deficits)

While this invention can be used in a wide range of settings like senior healthcare, and rehabilitation after traumatic brain injury or post neuro-skeletal surgery, its usage is particularly relevant for children in elementary education.

Several embodiments of this invention provide the option of using machine-learning technology for predictions. Additionally, the invention offers the ability to invoke traditional heuristics-based computational programs in parallel with the machine learning techniques. This enables comparison of results between traditional computation, that only analyses a subset of the data available through this invention, with results from a machine-learning-based analysis that uses almost all of the data captured.

What is claimed is:

1. A system for assessing visual perceptual, visual motor, and neuropsychological integration in humans, the system comprising:
a. a digital means to interact with human subjects and gather data through a client device by providing:
i. a set of visual forms,
ii. a goal-oriented task of reproducing the visual forms,
iii. a graphical user interface to allow the subject to complete the task,
iv. a mechanism to represent, measure, and capture the progression of the subject's thought during the process of completing that task,
v. a means to transfer captured data, which comprises multidimensional data points in multiple processable formats, to a data storage location or to a data processing system;
b. a data storage system that stores and archives captured data;
c. a data processing system that analyses the captured data using multiple computational methods, and classifies and evaluates the subject's visual cognitive function;
d. a reporting mechanism that compiles the classifications output by the data processing system and reports them to the subject.

2. A method for assessment of visual perceptual, visual motor, and neuropsychological function in humans, the method comprising:
a. capturing visual cognitive data that adequately represents one's ability and approach to solving visual problems, data such as—number of retries, speed of execution, direction, continuity or discontinuity of thought, separation of visual parts, visual-spatial awareness, general problem-solving approach, and
b. processing visual cognitive data using multiple computational methods that produce different classification metrics of visual cognitive function.

3. A non-transitory, computer-readable storage medium embodying computer program code, the computer program code comprising computer-executable instructions configured for:
a. providing a graphical user interface for measuring and capturing visual cognitive data,
b. storing captured data,
c. processing data use a plurality of computational approaches including:
i. traditional mathematical computation based on heuristics,
ii. Ensemble Machine Learning techniques comprising of unsupervised machine learning, supervised machine learning and transfer learning to analyze labeled image and numeric datasets,
iii. Unsupervised Machine Learning to analyze image and numeric datasets for discovery of new data features and characteristics.
